(19) United States
(12) Patent Application Publication
(10) Pub. No.: US 2009/0088854 A1
(43) Pub. Date: Apr. 2, 2009

(54) SPACING DEVICE AND METHOD

(75) Inventor: Kevin R. Strauss, Columbia, MD (US)

Correspondence Address: CARTER, DELUCA, FARRELL & SCHMIDT, LLP, 445 BROAD HOLLOW ROAD, SUITE 420, MELVILLE, NY, 11747, US

(73) Assignee: K2M, Inc., Leesburg, VA (US)

(21) Appl. No.: 12/240,001

(22) Filed: Sep. 29, 2008

Related U.S. Application Data
(60) Provisional application No. 60/995,591, filed on Sep. 27, 2007.
(60) Provisional application No. 60/999,310, filed on Oct. 17, 2007.

(51) Int. Cl. A61F 2/44; A61B 17/04

(52) U.S. Cl. 623/17.16; 623/17.11; 606/301

(57) ABSTRACT

A spinal device generally includes a spacing member and a fixing member. The spacing member is configured to maintain a space between adjacent vertebrae. The fixing member is adapted to maintain a position of the spacing member relative to the adjacent vertebrae. In addition, the fixing member includes a bendable portion releasably connected to the spacing member.

### CROSS-REFERENCE TO RELATED APPLICATIONS

The present application claims priority to, and the benefit of, U.S. Provisional Patent Application Ser. No. 60/995,591, filed on Sep. 27, 2007, and U.S. Provisional Patent Application Ser. No. 60/999,310, filed on Oct. 17, 2007. The entire contents of each provisional application cited above are hereby incorporated by reference.

### BACKGROUND

1. Technical Field

The present disclosure relates to devices and methods for orthopedic spine surgery. In particular, the present disclosure relates to devices for maintaining space between adjacent vertebrae and methods for implanting said devices.

2. Background of Related Art

The human spine is comprised of thirty-three vertebrae and twenty-four as an adult. An infant contains 7 cervical vertebrae, 12 dorsal or thoracic vertebrae, 5 lumbar vertebrae, 5 sacral vertebrae, and 4 coccygeal or caudal vertebrae. In an adult, the 5 sacral vertebrae fuse together to form the sacrum and the 4 coccygeal vertebrae fuse to form the coccyx. Invertebral discs lie between each pair of adjacent vertebrae. Every intervertebral disc maintains a space between adjacent vertebrae and acts as cushion under compressive, bending, and rotational loads and motions. Each intervertebral disc has a fibrocartilaginous central portion called the nucleus pulposus. The nucleus pulposus of a healthy intervertebral disc contains significant amount of water. This water content provides spongy quality and allows it to absorb spinal stress.

Each intervertebral disc has an annulus fibrosus, whose condition might be affected by the water content of the nucleus pulposus. The annulus fibrosus consist of a ring of fibrocartilage and fibrous tissue forming the circumference of the intervertebral disc. Excessive pressure or injuries to the intervertebral discs may adversely affect the annulus fibrosus. Usually, the annulus fibrosus is the first portion of the intervertebral discs that is injured. The annulus fibrosus may be injured in several ways. Typically, the annulus fibrosus tears due to an injury. When these tears heal, scar tissue forms in the annulus fibrosus. Given that scar tissue is not as strong as normal ligament tissue, the annulus becomes weaker as more scar tissue forms. An annulus fibrosus with scar tissue is usually weaker than a normal annulus fibrosus. The formation of scar tissue may eventually lead to damage to the nucleus pulposus. As a result of this damage, the nucleus fibrosus may, for instance, lose water content, hindering the intervertebral disc's ability to act as a cushion. The reduced cushioning capability might increase stresses on the annulus fibrosus and, consequently, cause still more tears. Hence, the annulus fibrosus may undergo a degenerative cycle consisting of exponential reduction of water content. Eventually, the nucleus pulposus may lose all its water. As the nucleus pulposus loses its water content, it collapses and thus allows the vertebrae above and below the disc space to move closer to each other. In other words, the intervertebral disc space narrows as the nucleus pulposus loses water. When the nucleus pulposus collapses, the facet joints, which are located on the back of the spine, shift, altering the way these joints work together.

When a disc or vertebrae is damaged due to disease or injury, the standard practice is to perform a spinal fusion. During spinal fusion, a surgeon removes part or all of the intervertebral disc, inserts a natural or artificial disc spacer, and constructs an artificial structure to hold the affected vertebrae in place. While the spinal fusion may address the diseased or injured anatomy, the natural biomechanics of the spine are affected in a unique and unpredictable way.

### SUMMARY

The present disclosure relates to a spinal device generally including a spacing member and a fixing member. The spacing member is configured to maintain a space between adjacent vertebrae. The fixing member is adapted to maintain the position of the spacing member relative to the adjacent vertebrae. In addition, the fixing member includes a bendable portion releasably connected to the spacing member.

The present disclosure further relates to a spinal device including a spacing member, a fixing member, and a fastening member. The spacing member is adapted to be positioned between adjacent vertebrae and is adapted to maintain a space between adjacent vertebrae. The fixing member is releasably connected to the spacing member and adapted to maintain the position of the spacing member relative to the adjacent vertebrae. The fastening member is configured to attach the fixing member to at least one of the adjacent vertebrae.

The present disclosure additionally relates to a method of maintaining a space between adjacent vertebrae. This method includes the step of providing a spacing device including a spacing member configured to maintain a space between adjacent vertebrae; positioning the spacing member between adjacent vertebrae; and fixing a position of the spacing member between the adjacent vertebrae.

### BRIEF DESCRIPTION OF THE DRAWINGS

Embodiments of the presently disclosed spacing device are disclosed herein with reference to the drawings wherein:

FIG. 1 is a perspective view of the an embodiment of the presently disclosed spacing device;

FIG. 2 is a top view of the spacing device of FIG. 1;

FIG. 3 is a front view of the spacing device of FIG. 1;

FIG. 4 is a side view of the spacing device of FIG. 1;

FIG. 5 is a side cross-sectional view of the spacing device of FIG. 1, taken along section line 5-5 of FIG. 3;

FIG. 6 is an anterior view of adjacent vertebrae with the spacing device of FIG. 1 disposed therebetween;

FIG. 7 is a lateral view of adjacent vertebrae with the spacing device of FIG. 1 disposed therebetween;

FIG. 8 is a perspective view of a further embodiment of the presently disclosed spacing device;

FIG. 9 is a top view of the spacing device of FIG. 8;

FIG. 10 is a front view of the spacing device of FIG. 8;

FIG. 11 is a side view of the spacing device of FIG. 8;

FIG. 12 is a side cross-sectional view of the spacing device of FIG. 8, taken along section line 12-12 of FIG. 10;

FIG. 13 is a lateral view of adjacent vertebrae with the spacing device of FIG. 8 disposed therebetween;

FIG. 14 is an anterior view of adjacent vertebrae with the spacing device of FIG. 8 disposed therebetween;

FIG. 15A is a top view of a compressible member according to an embodiment of the present disclosure;

FIG. 15B is a side view of the compressible member of FIG. 15A;

FIG. 15C is side cross-sectional view of the compressible member of FIG. 15A, taken along section line 15-15;

FIG. 16A is top view of a compressible member according to an embodiment of the present disclosure;

FIG. 16B is a side view of the compressible member of FIG. 16A; and

FIG. 16C is a side cross-sectional view of the compressible member of FIG. 16A, taken along section line 16-16 of FIG. 16B.

### DETAILED DESCRIPTION OF THE EMBODIMENTS

Embodiments of the presently disclosed spacing device will now be described in detail with reference to the drawings, wherein like reference numerals identify similar or identical elements. In the drawings and in the description that follows, the term “proximal” will refer to the end of a device that is closest to the operator, while the term “distal” will refer to the end of the device that is farthest from the operator. In addition, the term “cephalad” is used in this application to indicate a direction toward a patient's head, whereas the term “caudad” indicates a direction toward the patient's feet. Further still, for the purposes of this application, the term “medial” indicates a direction toward the middle of the body of the patient, whilst the term “lateral” indicates a direction toward a side of the body of the patient (i.e., away from the middle of the body of the patient). The term “posterior” indicates a direction toward the patient's back, and the term “anterior” indicates a direction toward the patient's front.

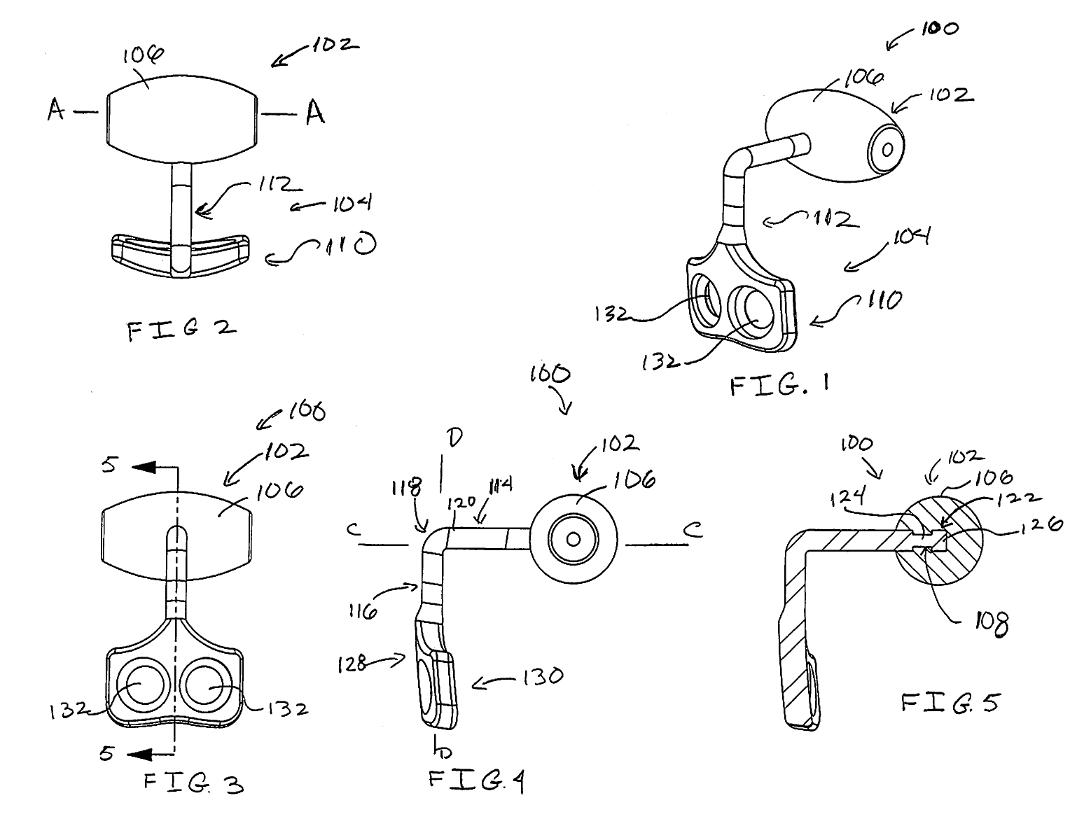

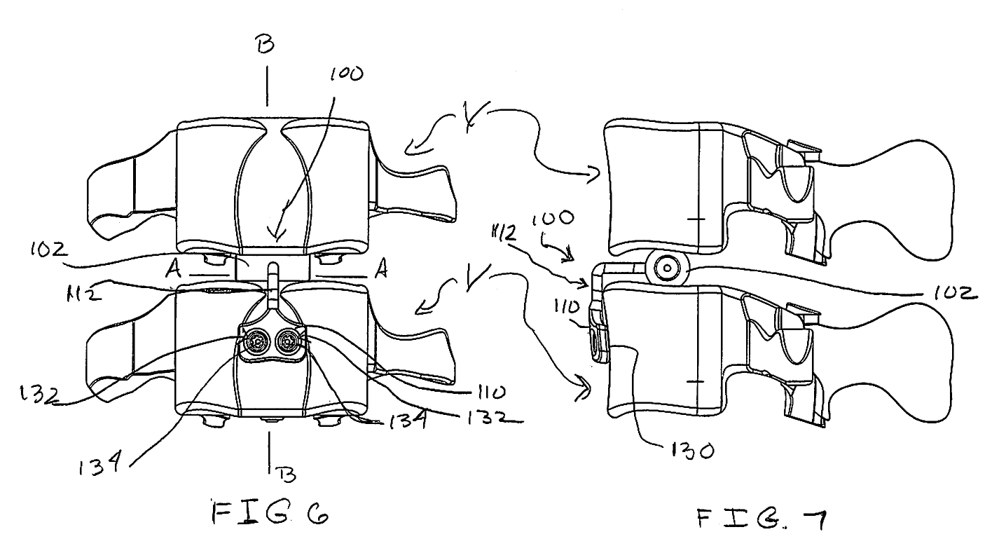

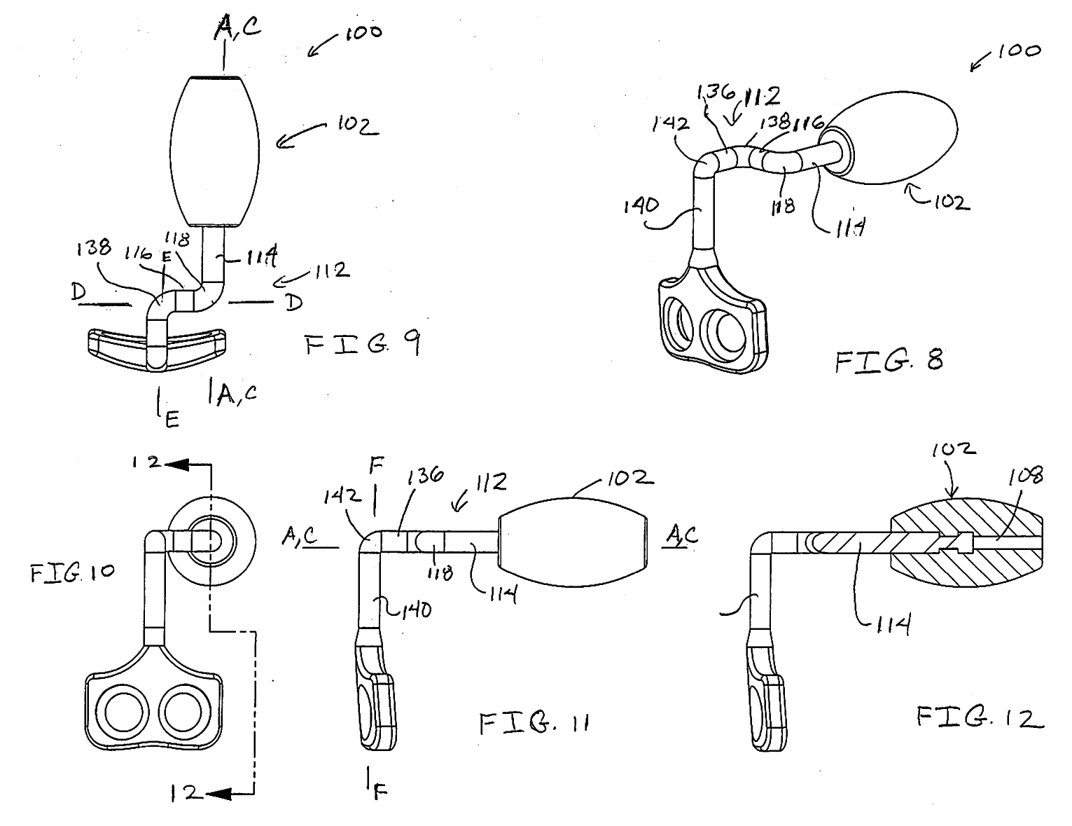

With reference to FIG. 1-7, a spacer device is generally designated with reference numeral 100. Spacer device 100 generally includes a spacing member 102 configured to maintain a space between adjacent vertebrae V and a fixing member 104 for attaching the spacing device 100 to at least one of the adjacent vertebrae V. Spacing member 102 may be made of a biocompatible material such as a polymer (e.g. polyamide, polyethylene, polyurethane, polyetheretherketone (PEEK), etc.), metal (titanium, titanium alloy, stainless steel, nickel titanium, cobalt chrome, etc.) ceramic, bone graft, composite or combinations thereof. Spacing member 102 may be made of any suitable material so long as the spacing member 102 can be compressed by the vertebrae V. In some embodiments, spacing member 102 defines a longitudinal axis A-A extending therethrough and has an oblong shape. When spacing member 102 is positioned between adjacent vertebrae V, longitudinal axis A-A is oriented substantially perpendicular to the long axis of the spine, which is represented by longitudinal axis B-B, as seen in FIG. 6. In these embodiments, spacing member 102 is compressed along longitudinal axis B-B when spacing member 102 is disposed between adjacent vertebrae V. Consequently, spacing member 102 allows translation of the vertebrae V along longitudinal axis B-B. Spacing member 102 further has a contact surface 106 for engaging the vertebral endplates during use. In operation, contact surface 106 has an area large enough to hinder the subsidence of adjacent vertebrae V, while allowing rotation of the vertebrae V about longitudinal axis B-B. In some embodiments, spacing member 102 is sized so that it only contacts the cortical rim and the softer, interior area of the vertebra endplate. Such spacing member 102 minimizes collapse between adjacent vertebrae V, while granting spacing member 102 a certain degree of freedom. As seen in FIG. 2, spacing member 102 defines a variable diameter along longitudinal axis A-A. This variable diameter facilitates rotation of the vertebrae about longitudinal axis B-B even when spacing member 102 is disposed between adjacent vertebrae V. In addition, spacing member 102 includes a longitudinal opening 108 dimensioned for receiving a portion of fixing member 104.

Fixing member 104 includes a plate 110 for attaching spacing device 100 to at least one vertebrae V and a bendable portion 112 for facilitating insertion of spacing device 100 inside a patient's body. Bendable portion 112 has an elongate shape. In certain embodiments, bendable portion 112 is semi-rigid. During the operation, a surgeon can bend bendable portion 112 while implanting spacer device 100 using either the standard posterior or transforaminal approaches. In various embodiments, bendable portion 112 is even more flexible and therefore allows spacing member 102 to translate or rotate relative to the plate 110.

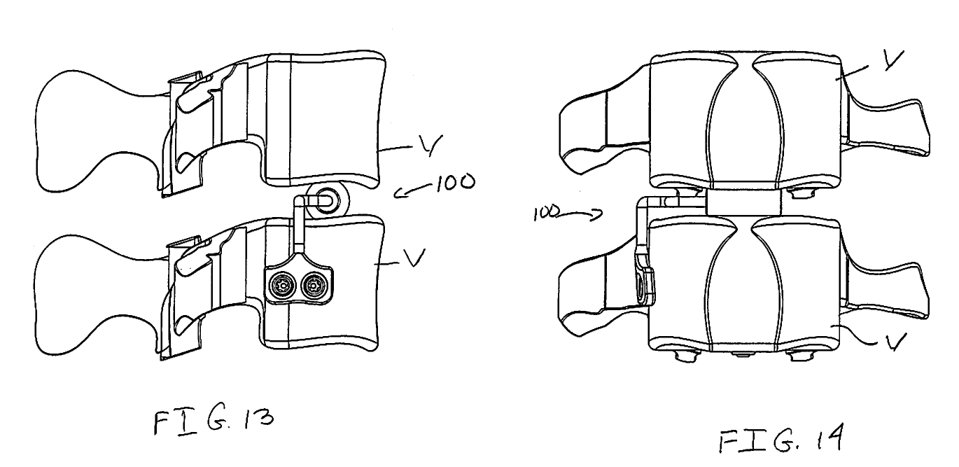

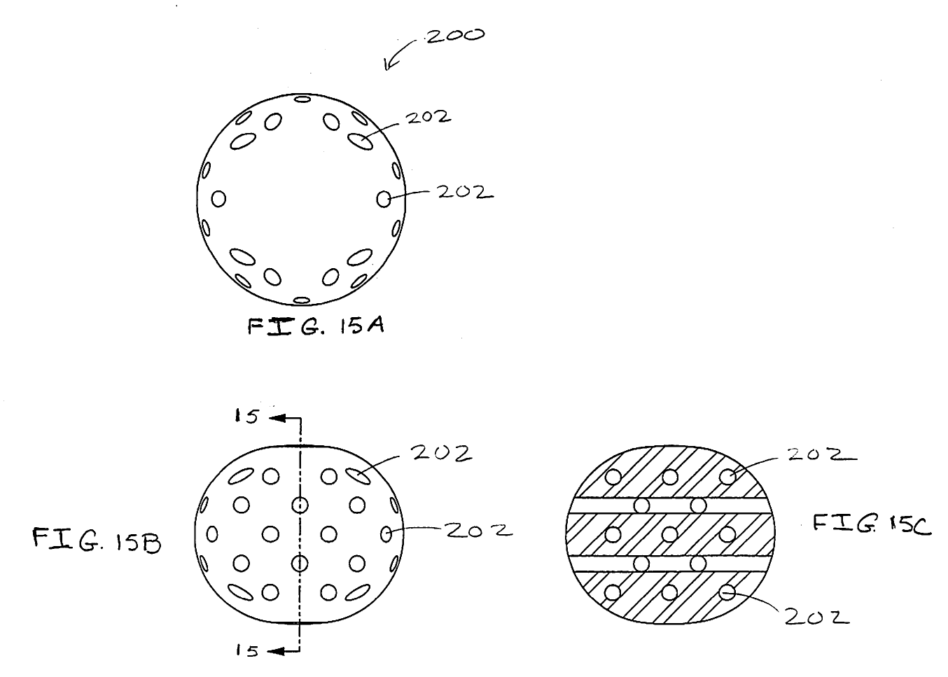

As seen in FIG. 4, bendable portion 112 includes a first section 114 defining a longitudinal axis C-C and a second section 116 defining a longitudinal axis D-D. Longitudinal axis C-C is substantially perpendicular to longitudinal axis D-D. Longitudinal axes C-C and D-D are substantially perpendicular to longitudinal axis A-A. A joint 118, such as an elbow, interconnects first and second sections 114, 116. First section 116 has a proximal end 120 fixed to joint 118 and a distal end 122 adapted to be received within longitudinal opening 108 of spacing member 102, as shown in FIG. 5. When distal end 122 of first section 114 is located inside longitudinal opening 108, distal end 122 and longitudinal opening 108 establish an interference fit, thereby releasably attaching spacing member 102 to fixing member 104. In certain embodiments, distal end 122 of first section 114 defines an annular recess 124 and an enlarged distal tip 126 located distally with respect to annular recess 124. Enlarged distal tip 126 and annular recess 124 collectively facilitate a releasable connection between spacing member 102 and fixing member 104.

Second section 116 of bendable portion 112 interconnects joint 118 and plate 110. Plate 110 has an exterior surface 128 and an inner surface 130 for engaging a vertebra V. In various embodiments, inner surface 130 has a contour mirroring the anterior section of a vertebra V, as seen in FIG. 7. In addition to the exterior and interior surfaces 128, 130, plate 110 includes at least one hole 132 adapted for receiving an anchoring or fastening member 134. In the embodiment depicted in FIG. 3, plate 110 has two holes 132. Plate 110 may nevertheless include more or fewer holes 132. As illustrated in FIG. 6, fastening members 134 may be pedicle screws or any suitable apparatus, means, or device capable of securing plate 110 to one or more vertebrae V. In operation, plate 110 is configured to be rigidly attached to at least one vertebra V, thereby fixing the position of spacing member 102 between adjacent vertebrae V.

During operation, the surgeon may employ a single spacing device 100 or multiple spacing devices. In any case, the geometry of spacing member 102 facilitates spinal fusion or dynamic stabilization. For example, spacing device 100 may be introduced between adjacent vertebrae, in conjunction with bone graft, to foster spinal fusion. The introduction of bone graft between adjacent vertebrae would likely change the form and/or the function of the vertebrae and consequently leads to changes in the vertebrae's internal architecture and in the external form, as predicated by Wolf's Law. Spacing device 100 may also be utilized with a posterior dynamic rod system.

Spacing device 100 may be built as a single unit. Alternatively, spacing device 100 may include several unattached pieces designed for assembly by a surgeon or healthcare professional. Moreover, spacing device 100 may have different sizes suitable for different anatomies. In certain embodiments, spacing device 100 is part of a kit. For instance, a kit may include several spacing devices 100 with different sizes. This kit provides the surgeon substantial surgical flexibility.

Referring to FIGS. 6-7, in operation, a surgeon initially achieves anterior exposure of the surgical site and then prepares the intervertebral space for the introduction of spacing device 100. To prepare the intervertebral space, the surgeon removes at least a portion of the intervertebral disc. A trial device may be inserted in the surgical site to determine the approximate size and target location of the spacing device 100. The surgeon subsequently places the spacing member 102 between adjacent vertebrae V and positions plate 110 next to vertebra V. During insertion of spacing device 100, the surgeon may bend bendable portion 112 to facilitate placement of spacing device 100. At any point during the procedure, the surgeon may confirm the position of spacing device 100 with radiographic images. Once the plate 110 is positioned on the anterior column of vertebra V, the surgeon introduces fastening members 134 through holes 132 and into vertebrae V, thereby securing plate 110 to vertebra V. Optionally, more than one spacing device 100 may be inserted in the patient. After insertion, the longitudinal axis A-A of spacing member 102 is oriented substantially orthogonal to the longitudinal axis B-B defined by the spine. Once spacing device has been implanted, the surgeon closes the surgical site. In an alternative surgical method, the surgeon accesses the surgical site using a quasi-lateral approach. In such case, the spacing device 100 is attached on a lateral side of the anterior column of the vertebral body, as seen in FIGS. 13 and 14.

Regardless of the specific method employed to implant spacing device 100, the surgeon may optionally perform a posterior procedure after inserting spacing device 100 to stabilize the posterior column of the spine. The posterior procedure may entail implanting bone anchoring members and spinal rods to provide posterior stabilization. In certain cases, the surgeon employs spinal rods made of a material that provides greater flexibility than conventional titanium alloy rods. By employing said spinal rods, the surgeon avoids completely compromising the patient's range of motion.

FIGS. 8-12 show a further embodiment of spacing device 100. Since the structure and operation of this embodiment is substantially similar to the structure and operation of the embodiment depicted in FIGS. 1-5, the present disclosure briefly describes this further embodiment. In this embodiment, longitudinal opening 108 spacing member 102 extends along longitudinal axis A-A. Longitudinal opening 108 is still adapted to receive first section 116 of bendable portion 114. First section 116 of bendable portion 114 defines longitudinal axis C-C. Longitudinal axis C-C, however, is oriented substantially parallel to longitudinal axis A-A of spacing member 102. Joint 118 interconnects first and second sections 114, 116 of bendable portion 112. Second section 116 still defines longitudinal axis D-D oriented substantially perpendicular to longitudinal axis C-C of first section 114.

Bendable portion 112 additionally includes a third section 136 defining a longitudinal axis E-E. Longitudinal axis E-E is substantially parallel to longitudinal axes A-A and C-C, but substantially orthogonal to longitudinal axis D-D. A joint 138, such as an elbow, interconnects second and third sections 116, 136. Further, bendable portion 112 includes a fourth section 140 connected to third section 136 by a joint 142. Fourth section 140 defines a longitudinal axis F-F oriented substantially perpendicular to longitudinal axes A-A, C-C, D-D, and E-E, as illustrated in FIG. 11. Plate 110 is coupled to fourth section 140 of bendable portion 112.

With reference to FIGS. 13 and 14, the surgeon may implant the embodiment of spacing device 100 shown in FIGS. 8-12 by employing a lateral approach. In this case, spacing member 102 is inserted laterally between adjacent vertebrae V and plate 110 is attached on a lateral side of the anterior column of the vertebral body.

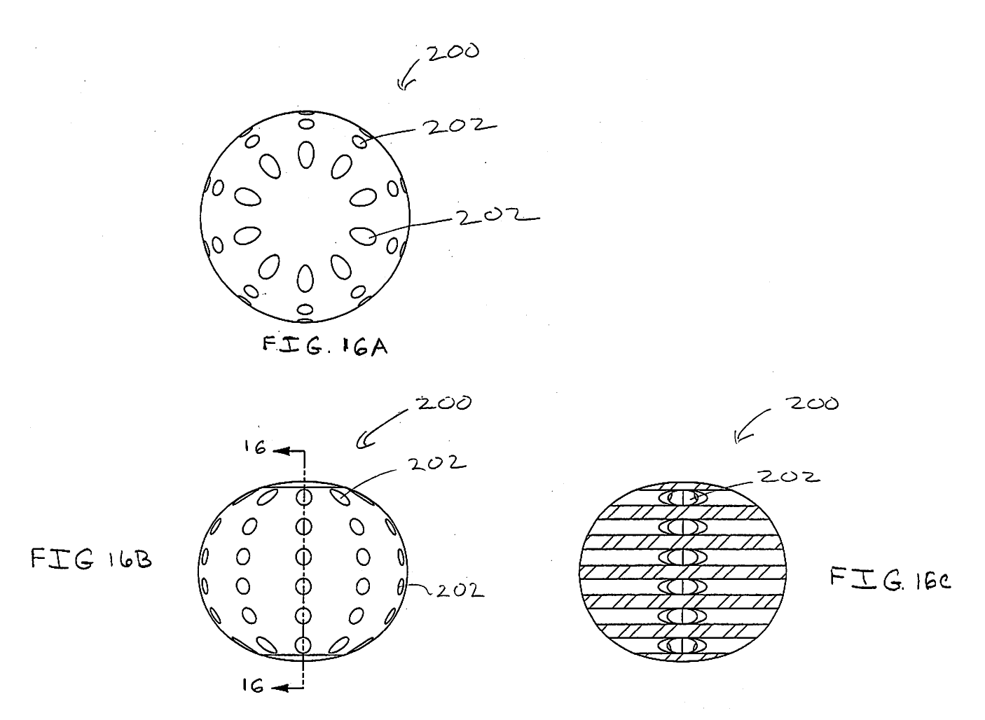

With reference to FIGS. 15A-16C, alternative embodiments of the spacing members 200 are shown in several views. The spacing member 200 is preferentially made from a biocompatible polymeric material but may be made from any other biocompatible material, including metal, if features are included to allow the device to be compressed along the long axis of the spine when implanted between adjacent vertebrae. The spacing member 200 has sufficient contact surface area with a vertebral body endplate such that minimal subsidence into the endplate occurs and rotation about the long axis of the spine is possible. The dual direction radii of the spacing member's 200 outer surface provide articulating surfaces whereby rotation of the vertebrae about the other two axes may be achieved. Spacing member 200 includes holes 202.

The spacing member 200 is also contemplated as a single, central vertebral body spacing device that may be used alone or with posterior support. When using posterior support, the spacing member 200 may facilitate a fusion or dynamic stabilization procedure. The former takes advantage of Wolff's Law when bone graft is introduced. The latter may be used with a posterior dynamic system. A range of sizes will be made available to better fit the patients' anatomy and offer greater surgical flexibility.

Still another embodiment is contemplated wherein a rigid member is suspended on the interior of the device such that when the device is compressed a specified distance no further compression may occur due to the limitations provided by the rigid member. The rigid member is preferentially made of titanium but may be manufactured from any other biocompatible, non-compressible material.

A method for use is also disclosed herein for proper implantation of the device. Initially, the disc space is accessed either through an anterior, lateral or posterior approach. A channel is cut into the disc space and the appropriate amount of material is removed. A trial device is then introduced for appropriate sizing and placement. At least one spacing member 200 is then inserted through the channel into the void in the disc space. Radiographic images are taken to ensure proper alignment of the device and the channel is blocked to prevent expulsion of spacing member 200. When spacing member 200 is used in conjunction with a posterior stabilizing spinal construct, bone anchoring means and spinal rods are implanted. The spinal rods are preferentially more flexible than a traditional titanium alloy rod such that the patient's range of motion is not completely compromised. After the spinal rods are secured, the surgical site is closed and the patient may begin recovery.

It will be understood that various modifications may be made to the embodiments of the presently disclosed spacing devices. Therefore, the above description should not be construed as limiting, but merely as exemplifications of embodiments. Those skilled in the art will envision other modifications within the scope and spirit of the present disclosure.

What is claimed is:

1. A spinal device, comprising:
a spacing member configured to maintain a space between adjacent vertebrae; and
a fixing member adapted to maintain a position of the spacing member relative to the adjacent vertebrae, the fixing member including a bendable portion releasably connected to the spacing member.

2. The spinal device of claim 1, wherein the bendable portion is semi-rigid.

3. The spinal device of claim 1, wherein spacing member is compressible.

4. The spinal device of claim 1, wherein the fixing member includes a plate adapted to be rigidly fixed to at least one of the adjacent vertebrae.

5. The spinal device of claim 4, wherein the plate includes a first hole configured to receive a first fastening member.

6. The spinal device of claim 5, wherein the plate includes a second hole configured to receive a second fastening member.

7. The spinal device of claim 6, wherein at least one of the first and second fastening members is a screw.

8. The spinal device of claim 1, wherein the spacing member is at least partially made of a biocompatible material.

9. The spinal device of claim 1, wherein the spacing member has an oblong shape.

10. A spinal device, comprising:
a spacing member adapted to be positioned between adjacent vertebrae, wherein the spacing member is adapted to maintain a space between adjacent vertebrae;
a fixing member releasably connected to the spacing member, the fixing member being adapted to maintain a position of the spacing member relative to the adjacent vertebrae; and
a fastening member configured to attach the fixing member to at least one of the adjacent vertebrae.

11. The spinal device of claim 10, wherein the fastening member is a pedicle screw.

12. The spinal device of claim 10, wherein the spacing member has an oblong shape.

13. The spinal device of claim 10, wherein the fixing member includes a bendable portion releasably coupled to the spacing member and a plate attached to the bendable portion.

14. The spinal device of claim 13, wherein the bendable portion is semi-rigid.

15. The spinal device of claim 13, wherein the plate includes a hole adapted to receive the fastening member.

16. The spinal device of claim 13, wherein the bendable portion has an elongate shape.

17. The spinal device of claim 13, wherein the bendable portion has a first section defining a first longitudinal axis and a second section defining a second longitudinal axis, the first longitudinal axis being substantially orthogonal to the second longitudinal axis.

18. The spinal device of claim 17, wherein the bendable portion includes a third section defining a third longitudinal axis, wherein the third longitudinal axis is oriented substantially parallel to the first longitudinal axis.

19. A method of maintaining a space between adjacent vertebrae, comprising the steps of:
providing spacing device including a spacing member configured to maintain a space between adjacent vertebrae;
positioning the spacing member between adjacent vertebrae; and
fixing a position of the spacing member between the adjacent vertebrae.

20. The method of claim 19, wherein the step of fixing the position of the spacing member includes rigidly attaching a portion of the spacer device to at least one of the adjacent vertebrae.
